(19) United States
(12) Patent Application Publication
(10) Pub. No.: US 2023/0251274 A1
(43) Pub. Date: Aug. 10, 2023

(54) PTX3 AS PROGNOSTIC MARKER IN COVID-19

(71) Applicants: HUMANITAS MIRASOLE S.P.A., Rozzano (MI) (IT); HUMANITAS UNIVERSITY, Pieve Emanuele (MI) (IT)

(72) Inventors: Barbara BOTTAZZI, Pieve Emanuele (MI) (IT); Cecilia GARLANDA, Pieve Emanuele (MI) (IT); Alberto MANTOVANI, Pieve Emanuele (MI) (IT)

(73) Assignees: HUMANITAS MIRASOLE S.P.A., Rozzano (MI) (IT); HUMANITAS UNIVERSITY, Pieve Emanuele (MI) (IT)

(21) Appl. No.: 18/003,064

(22) PCT Filed: Jun. 25, 2021
(86) PCT No.: PCT/EP2021/067492

§ 371 Date: Dec. 22, 2022

(30) Foreign Application Priority Data
Jun. 25, 2020 (EP) 20182181.6

(51) Int. Cl. G01N 33/68

(52) U.S. Cl. CPC G01N 33/6893; G01N 2800/52; G01N 2800/56; G01N 2333/4715

(57) ABSTRACT

The present invention refers to an in-vitro or ex vivo method for the prognosis of a Coronavirus disease and/or for the monitoring of the efficacy of a therapeutic treatment of a Coronavirus disease having the steps of: a) detecting and/or measuring the amount of the protein PTX3 or of fragments thereof or of the polynucleotide coding for the protein or of fragments thereof in an isolated biological sample obtained from the subject Preferably the method further includes—the step: b) comparing the same with a proper control.

### TECHNICAL FIELD

The present invention refers to an in-vitro or ex vivo method for the prognosis of a Coronavirus disease and/or for the monitoring of the efficacy of a therapeutic treatment of a Coronavirus disease comprising the steps of detecting and/or measuring the amount of the protein PTX3 or of fragments thereof or of the polynucleotide coding for said protein or of fragments thereof in an isolated biological sample obtained from the subject.

### BACKGROUND ART

Highly pathogenic betacoronaviruses, causing Severe Acute Respiratory Syndrome (SARS), Middle East Respiratory Syndrome (MERS), and the currently pandemic COVID-19, affect the lower respiratory tract leading to critical acute respiratory distress syndrome (ARDS) and fatality in a high percentage of cases¹⁻⁴. SARS-CoV-2 infection is characterized by variable clinical forms with symptoms including fever, cough, and general malaise in mild and moderate cases⁵, which progress to severe pneumonia or ARDS, shock and/or multiple organ failure, requiring hospitalization in Intensive Care Units (ICU), in more severe cases. The high morbidity and mortality observed in COVID-19 pandemics are caused by alveolar damage and pneumonia^{6,7}, cardiovascular complications, and multiorgan failure⁸.

SARS-CoV-2 interacts with ACE2 expressed by pneumocytes in the alveolar lining, leading to lung injury. ACE2 is also widely expressed on endothelial cells, thus possibly explaining the evidence of direct viral infection of the endothelium, diffuse endothelial inflammation, and widespread microvascular dysfunction, leading to organ ischaemia, inflammation, edema, and a procoagulant state^{4,9,10}. In addition, uncontrolled activation of innate and adaptive immunity in response to the infection results in hyperinflammatory responses, as demonstrated by the cytokine storm and activation of macrophages and neutrophils in COVID-19 patients, which, by affecting lung tissue and blood vessels, contribute to ARDS pathogenesis, shock and multiorgan failure^{4,7}.

PTX3 is a key component of humoral innate immunity, belonging to the family of pentraxins¹¹. In contrast with its relative, the short pentraxin C reactive protein (CRP), essentially produced by the liver in response to IL-6 during the acute phase response, PTX3 is rapidly produced by several cell types, including myeloid cells, endothelial cells, and respiratory epithelial cells, in particular in response to IL-1, TNFα, microbial molecules, and tissue damage¹¹. PTX3 is an essential component of humoral innate immunity, involved in resistance to selected pathogens and in the regulation of inflammation.

The similarity with CRP prompted investigation of the usefulness of PTX3 as a marker in diverse human conditions of infective or inflammatory origin. The local production by different cell types at inflammatory sites and the release of the preformed protein by neutrophils in response to primary proinflammatory cytokines or microbial recognition accounts for the rapidity of PTX3 increase in these conditions. Increased PTX3 plasma concentrations were described in infections of fungal, bacterial and viral origin¹²⁻¹⁵, severe inflammatory response syndrome (SIRS) and sepsis¹⁶⁻¹⁸, and cardiovascular diseases¹⁹⁻²¹. In different pathological conditions high PTX3 plasma levels were associated with disease severity and mortality (e.g.¹⁶⁻¹⁸).

Moreover, PTX3 has been shown to serve as a biomarker of disease activity in inflammatory conditions involving the vascular bed, ranging from atherosclerosis to vasculitis (e.g.^{19,22-27}).

Previous findings on the prognostic significance of PTX3 in systemic inflammatory conditions as well as in vascular pathology^{16,26} prompted the present investigation which was designed to assess PTX3 expression and plasma levels as a biomarker in COVID-19.

### SUMMARY OF THE INVENTION

Pentraxin 3 (PTX3) is a fluid phase pattern recognition molecule produced by different cell types, in particular macrophages and vascular cells. PTX3 plasma levels are associated with poor outcomes in systemic inflammatory conditions and vascular pathology. The present study was designed to assess expression and significance of PTX3 as a biomarker in COVID-19 considering short-term mortality at 28 days after hospitalization as the primary outcome.

Here inventors report that, based on a bioinformatics analysis on public databases, PTX3 was strongly induced by SARS-COV-2 in respiratory tract epithelial cells and that at the single cell level, COVID-19 monocytes and lung macrophages expressed PTX3. Finally, PTX3 plasma levels are a strong predictor of 28-day mortality in hospitalized COVID-19 patients.

Indeed, prompted by these data and by previous results in systemic inflammatory conditions, PTX3 plasma levels were measured in a cohort of 96 consecutive COVID-19 patients admitted to Humanitas Clinical and Research Center, Milan (Italy). PTX3 plasma concentrations were significantly higher in patients admitted in ICU compared with patients in standard wards (median 21.0 ng/mL, IQT 15.5-46.3 ng/mL and 12.4 ng/mL IQT 6.1-20.2 ng/mL, respectively; p=0.0017), and significantly higher in patients who died (median, 39.79 ng/mL, IQT 20.2-75.7 ng/mL, and 15.3 ng/mL, IQT 8.2-21.3 ng/mL in survivors, respectively; p=0.0001). After determining an optimal PTX3 cut off for our primary outcome, the Kaplan-Meier curve showed an overall 28-day event-free survival of 0.94±0.03 (95% CI, 0.83 to 0.97) in patients with PTX3<22.25 ng/mL and 0.52±0.08 (95% CI, 0.34 to 0.67) in patients with PTX3≥22.25 ng/mL. In Cox regression model, PTX3 levels were associated with increased mortality rate at 28 days after hospitalization in COVID-19 with an adjusted Hazard Ratio of 7.8 (95% CI 2.54-24.03). C-reactive protein was significantly associated to high basal SOFA score (adjusted HR 1.14; 95% CI 1.05-1.23), but was not confirmed as a predictor of mortality.

The results reported here suggest that PTX3 is induced by SARS-CoV-2 in lung epithelial cells and is expressed in monocytes and lung macrophages as analyzed at single cell level. PTX3 plasma levels can serve as a strong prognostic indicator of short-term mortality in COVID-19.

It is therefore an object of the invention an in-vitro or ex vivo method for the prognosis of a Coronavirus disease and/or for the monitoring of the efficacy of a therapeutic treatment of a Coronavirus disease comprising the steps of:

a) detecting and/or measuring the amount of the protein PTX3 or of fragments thereof or of the polynucleotide coding for said protein or of fragments thereof in an isolated biological sample obtained from the subject,

preferably the method further comprises the step:

b) comparing the same with a proper control.

Preferably, the Coronavirus is a beta Coronavirus, preferably SARS-CoV-2, and/or the Coronavirus disease is selected from the group consisting of: Severe Acute Respiratory Syndrome (SARS), Middle East Respiratory Syndrome (MERS), COVID-19, coronavirus-associated acute respiratory distress syndrome (ARDS).

Preferably, the biological sample is selected from the group consisting of: plasma, serum, blood, CSF, saliva, or Bronchoalveolar lavage fluid (BALF), pulmonary tissue.

Preferably, the amount of PTX3 is detected or measured by means of specific antibody or coulometric or electrochemical detector.

Preferably, the subject is a patient who has been or was diagnosed with a Coronavirus disease. Pulmonary tissue may be obtained by way of biopsy on a diseased or deceased patient.

In a preferred embodiment when PTX3 is higher than the proper control, the subject is at risk of short-term mortality or of being affected by a more severe disease and/or of a poor prognosis.

The proper control is preferably a cut-off value.

In a preferred embodiment when PTX3 levels, preferably in a plasma sample, are equal or above about 20 or about 21 or about 22 ng/mL, the subject is at risk of short-term mortality and/or of being affected by a more severe disease and/or of a poor prognosis.

In a preferred embodiment when PTX3 levels in a plasma sample are equal or above 22.25 ng/mL, the subject is at risk of short-term mortality and/or of being affected by a more severe disease and/or of a poor prognosis.

Another object of the invention is the biomarker PTX3 for use in a method for the prognosis of a Coronavirus disease and/or for the monitoring of the efficacy of a therapeutic treatment of a Coronavirus disease.

A further object of the invention is the biomarker PTX3 for use in a method for the prognosis of a Coronavirus disease and/or for the monitoring of the efficacy of a therapeutic treatment of a Coronavirus disease, said method being as defined above.

Another object of the invention is the use of the biomarker PTX3 in a method for the prognosis of a Coronavirus disease and/or for the monitoring of the efficacy of a therapeutic treatment of a Coronavirus disease.

Preferably, the Coronavirus is a beta Coronavirus, preferably SARS-CoV-2, and/or the Coronavirus disease is selected from the group consisting of: Severe Acute Respiratory Syndrome (SARS), Middle East Respiratory Syndrome (MERS), COVID-19, coronavirus-associated acute respiratory distress syndrome (ARDS).

Another object of the invention is a kit for the prognosis of a Coronavirus disease and/or for the monitoring of the efficacy of a therapeutic treatment of a Coronavirus disease comprising:
means to detect and/or measure the amount of at least the biomarker PTX3 and optionally
control means.

Another object of the invention is a kit according to the invention for use in the method as defined above or the use of said kit in the method as defined above.

A further object of the invention is the use of a kit comprising:
means to detect and/or measure the amount of at least the biomarker PTX3 and optionally
control means.
for carrying out the method according to the invention.

Preferably the kit is the kit of the invention.

### DETAILED DESCRIPTION OF THE INVENTION

By “coronavirus infection” or “coronavirus disease” it is intended an infection or disease caused by or otherwise associated with growth of coronavirus in a subject, in the family Coronaviridae (subfamily Coronavirinae).

Preferably, the coronavirus is selected from the group consisting of:

(a) alphacoronavirus;

(b) betacoronavirus;

(c) gammacoronavirus;

(d) deltacoronavirus; and

(e) coronavirus which causes Acute respiratory distress syndrome (ARDS).

Examples of coronaviruses that cause ARDS are MERS-CoV, SARS-CoV, SARS-CoV-2, HCoV-NL63

Examples of alphacoronaviruses include Alphacoronavirus 1, Bat coronavirus CDPHE15, Bat coronavirus HKU10, Human coronavirus 229E, Human coronavirus NL63, Miniopterus bat coronavirus 1, Miniopterus bat coronavirus HKU8, Mink coronavirus 1, Porcine epidemic diarrhoea virus, Rhinolophus bat coronavirus HKU2, and Scotophilus bat coronavirus 512.

Examples of betacoronaviruses include Murine coronavirus, Betacoronavirus 1, Hedgehog coronavirus 1, Human coronavirus HKU1, Middle East respiratory syndrome-related coronavirus, Pipistrellus bat coronavirus HKU5, Rousettus bat coronavirus HKU9, Severe acute respiratory syndrome-related coronavirus, and Tylonycteris bat coronavirus HKU4.

Examples of gammacoronaviruses include Avian coronavirus and Beluga whale corona virus SW1. Examples of deltacoronaviruses include Bulbul coronavirus HKU11, Common moorhen coronavirus HKU21, Coronavirus HKU15, Munia coronavirus HKU13, Night heron coronavirus HKU19, Thrush coronavirus HKU12, White-eye coronavirus HKU16, and Wigeon coronavirus HKU20.

Preferably, the coronavirus is a human coronavirus, more preferably it is selected from the group consisting of:

(a) human coronavirus 229E;

(b) human coronavirus OC43;

(c) Severe Acute Respiratory Syndrome coronavirus (SARS-CoV)

(d) human Coronavirus NL63 (HCoV-NL63, New Haven coronavirus);

(e) human coronavirus HKU1;

(f) Middle East respiratory syndrome coronavirus (MERS-CoV);

(g) Severe Acute Respiratory Syndrome coronavirus 2 (SARS-CoV-2).

Preferably the nucleotide sequence and amino acidic sequence of PTX3 is as defined in GeneID: 5806; X63613; NCBI Reference Sequence: NP_002843.2

More preferably PTX3 is the full length protein or it doesn't comprise the signal peptide. PTX3 may comprise or consists of the N-terminal region and/or of the C-terminal region thereof.

Said detection and/or measurement of the amount of the protein PTX3 or of fragments thereof is preferably carried out by an immunoassay, preferably an ELISA assay, more preferably a sandwich ELISA.

The in vitro or ex vivo method according to the invention preferably comprises the steps of:

a) contact and incubation of the biological sample with the above defined antibody thereby forming a PTX3-antibody complex, if PTX3 is present;

b) separation of the biological sample from the PTX3-antibody complex;

c) selective detection of PTX3 bound to the antibody and/or quantifying the amount of PTX3 bound to the antibody using detecting means for the antibody;

d) comparison of the result obtained in c) to a control result.

Preferably the antibody is immobilized to a solid support, more preferably the immobilized antibody being coated on a plate, preferably a microtiter plate.

The detecting means is preferably a detectable antibody. A detectable antibody is preferably directly detectable, optionally the detectable antibody is amplified by a fluorimetric reagent, further optionally wherein the detectable antibody is biotinylated, and the detection means is avidin or streptavidin-peroxidase and 3,3′,5,5′-tetramethyl benzidine or the detectable antibody is conjugated to alkaline phosphatase, and the detection means is p-nitrophenyl phosphate and/or 4-methylumbelliferyl phosphate.

Said detection and/or measurement of the amount of the protein PTX3 or of fragments thereof is preferably carried out by an ELISA assay.

Preferably the kit of the invention further comprises a solid support wherein the antibody is immobilized, said solid support being preferably a microtitration plate.

Preferably the above defined method is an immunoassay, more preferably an ELISA, and/or it detects and/or quantifies the human PTX3 in the biological sample.

All known antibodies recognizing PTX3 may be used, e.g.: 16B5 (Bottazzi et al., J. of Biol. Chem. 272(52), 32817-23, 1997); 1C8 (Bottazzi et al., J. of Biol. Chem. 1997, 272(52), 32817-32823); MNB4 (Peri et al., Circulation 2000, 102, 636-641); MNB6 (Peri et al., Circulation 2000, 102, 636-641); MNB10 (WO2005/106494); Pen-3 (WO2005/106494); PPMX0101 (WO2005/080981); PPMX0102 (WO2005/080981); PPMX0112 (WO2005/080981); and PPMX0148 (WO2005/080981). Preferably the antibody is murine monoclonal antibody MNB4 and the detection antibody used in the present invention is a biotinylated rabbit antiserum (pAb) raised against human PTX3 (Knoflach et al., PloseOne 2012).

A typical immunoassay according to the invention comprises the stages of i) exposing biological plasma sample to a monoclonal or polyclonal antibody, preferably a monoclonal antibody, against PTX3, and ii) determining specific antigen/antibody binding. Antigen/antibody binding may be measured using radioisotopic or non-radioisotopic methods. For example, these include IRMA (Immune Radioimmunometric Assay), EIA (Enzyme Immuno Assay), ELISA (Enzyme Linked Immuno Assay), FIA (Fluorescent Immuno Assay) and CLIA (Chemiluminescent Immune Assay) techniques. The method envisages the use of means of detection such as radionuclide labels, enzymes, coenzymes, fluorophores, chemiluminescence agents, chromogens, substrates, enzyme cofactors or inhibitors, the production of free-radicals, dyes etc. In a preferred embodiment, the immunoassay is an ELISA-type assay and envisages use of the rat monoclonal antibody MNB4 (IgG2a), capable of binding specifically to PTX3 with a sensitivity of less than 100 pg/ml, and a rabbit anti-PTX3 polyclonal antibody (biotinylated IgG).

In the context of the present invention the reference to “PTX3” may indicate the amount or levels of the protein PTX3 or of fragments thereof or of the polynucleotide coding for said protein or of fragments thereof. In the above methods, an amount of said protein PTX3 or of fragments thereof or of said polynucleotide or of fragments thereof in the isolated biological sample obtained from the subject higher than the control amount indicates that the subject will present a more severe disease and/or indicates a poor prognosis.

In the present invention, a proper control may be selected from a PTX3 reference value measured in a healthy patient, a patient affected by a non-coronavirus pathology or who is not affected by a Coronavirus disease.

Preferably, said PTX3 reference value or proper control or control amount is equal to the level or amount of plasma PTX3 that can be determined in healthy individuals and is preferably equal to 2 ng/ml, more preferably 5 ng/ml PTX3.

The isolated biological sample of the above defined methods is preferably plasma, blood, serum, bronchoalveolar lavage fluid, pulmonary tissue, cells culture, cell supernatants, cell lysates, tissue samples, organs, bone marrow. In other embodiments of the invention a device or kit is provided for the analysis of patient samples. Alternatively, the reagents can be provided as a kit comprising reagents in a suspension or suspendable form, e.g. reagents bound to beads suitable for flow cytometry, preferably magnetic beads coated with antibody capture, or customized dried antibody cocktails for Multicolor analysis and/or columns with sized filter cartridges, and/or combined with specific antibody filter (SAF) and the like. The instructions may comprise instructions for conducting an antibody-based flow cytometry assay. Detecting means are preferably means able to detect and/or measure the amount of PTX3, e.g. means able to detect the complex antigen-antibody, as enzyme conjugated secondary antibodies, luminescent substrates, magnetic beads coated with antibody capture, customized dried antibody cocktails and/or columns with size filter cartridges and/or combined with specific antibody filter (SAF). The kit of the invention preferably comprises anti-PTX3 monoclonal and polyclonal antibodies. Preferably, said antibodies will be a rat monoclonal antibody MNB4 (IgG2a) and a rabbit polyclonal antibody (biotinylated IgG).

Optionally, the kit of the invention may comprise recombinant human PTX3 and/or detection reagents and/or buffers and/or diluents and/or stabilizers and/or other reagents of use in performing the immunoassay, in joined or separate containers. Preferably said kit further comprises a solid support wherein the antibody is immobilized. Preferably, the kit of the invention is an immunoassay kit, more preferably an ELISA kit. The means for detecting and/or quantifying the antigen-antibody complex may be means for detecting and/or quantifying the catalytic activity of PTX3 such as secondary antibodies conjugated to enzymes, luminescent substrates. These means are known in the art. The kit according to the invention can further comprise typical auxiliary components, such as buffers, carriers, dyes, etc. and/or instructions for use. The kit can further comprise control means for comparing the increase in the amount of PTX3 with an appropriate control value (or reference value or proper control). The control value can be obtained, for example, with reference to known standards, either from a normal subject or a normal population. In the present invention, the “control result” (or control value or reference value or proper control) can be the result obtained for a sample isolated from a healthy subject or from a patient affected by another disorder than coronavirus disease.

In the context of the present invention for “risk of short-term mortality” it is e.g. intended a higher risk to die within 4 weeks with respect to other patients affected by the same coronavirus disease.

The in vitro method for monitoring the efficacy of a therapeutic treatment, as defined above, preferably comprises the steps of:

a) detecting and/or measuring the amount or alteration of the protein PTX3 or of fragments thereof or of the polynucleotide coding for said protein or of fragments thereof in an isolated biological sample obtained from the subject and

b) comparing the measured amount or alteration of step a) with a proper control.

In the present invention, a proper control may be selected from a PTX3 reference value (or control value) measured in a healthy patient, a patient affected by a non-coronavirus pathology or who is not affected by Coronavirus disease, a patient affected by Coronavirus disease before a therapeutic treatment, or during the time course of a therapeutic treatment, a or at various time points during the course of the disease.

In the case of a method for monitoring the efficacy of a therapeutic treatment, the control sample can be a sample taken from the same subject prior to the start-up of the therapy or taken at various times during the therapy. By “monitoring the efficacy” it is meant monitoring the pharmacological profile of an agent. In the case of a method for screening a treatment of Coronavirus diseases, the control sample can be a sample taken from an untreated subject or a subject treated with a substance to be tested or a subject treated with a reference treatment.

In the context of the present invention, the optimal cut-off value may be obtained using receiver operating characteristic (ROC) curves. Therefore, the cut-off value or the control value or the proper control may be obtained easily by the skilled man. In a preferred embodiment, it corresponds to 20, 21, 22, 22.1, 22.2, 22.21, 22.22, 22.23, 22.24, 22.25, 22.3, 22.4, 23, 24 or 25 ng/mL, more preferably it corresponds to 22.25 ng/mL. Alternatively, it may be lower, e.g. it may correspond to 9, 10, 11, 12, 13, 14, 15, 16, 17, 18 or 19 ng/mL. Indeed, the cut-off value may depend on different clinical characteristics (more or less serious disease, different ethnicity and genetic background, previous or concomitant therapies) of the patient.

In a preferred embodiment when PTX3 levels, preferably in a plasma sample, are equal or above about 9, 10, 11, 12, 13, 14, 15, 16, 17, 18, 19, 20, 21, 22, 22.1, 22.2, 22.21, 22.22, 22.23, 22.24, 22.25, 22.26, 22.3, 22.4, 23, 24 or 25 ng/mL, the subject is at risk of short-term mortality and/or of being affected by a more severe disease and/or of a poor prognosis.

In the context of the present invention for “risk of being affected by a more severe disease” it is e.g. intended a higher risk of needing intensive care treatment with respect to other patients affected by the same coronavirus disease.

In the present invention, the expression “detect” or “detection” refers to any use of any method of observation, assessment or quantification of the signals indicative of the antibody's presence in a sample or the absolute or relative amount of said antibody in a sample, for example by chemiluminescence, fluorimetry, spectrophotometry, etc. In the present invention, the expression “quantify” or “quantification” can be understood as measuring the amount or concentration or level of PTX3 or of the respective antibody, preferably with a semi-quantitative or quantitative method. The term “amount”, as used in the description refers to but is not limited to the absolute or relative amount of proteins, and any other value or parameter associated with the latter or which can derive therefrom. Such values or parameters comprise signal intensity values obtained either for physical or chemical properties of the protein, obtained by direct measurement, for example, intensity values in an immunoassay, mass spectroscopy or nuclear magnetic resonance. Moreover, these values or parameters include the ones obtained by indirect measurement.

When describing the present invention, all terms not defined herein have their common art-recognized meanings. Any term or expression not expressly defined herein shall have its commonly accepted definition understood by those skilled in the art. To the extern that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

The present invention also includes functional fragments, variants or derivatives of the proteins, peptides or sequences herein disclosed. In the context of the present invention, when referring to specific DNA sequences, it is intended that it is comprised within the invention also RNA molecules identical to said polynucleotides, except for the fact that the RNA sequence contains uracil instead of thymine and the backbone of the RNA molecule contains ribose instead of deoxyribose, RNA sequence complementary the sequences therein disclosed, functional fragments, mutants and derivatives thereof, proteins encoded therefrom, functional fragments, mutants and derivatives thereof. The term “complementary” sequence refers to a polynucleotide which is non-identical to the sequence but either has a complementary base sequence to the first sequence or encodes the same amino acid sequence as the first sequence. A complementary sequence may include DNA and RNA polynucleotides. The term “functional” may be understood as capable of maintaining the same activity. “Fragments” are preferably long at least 10 aa., 20 aa., 30 aa., 40 aa., 50 aa., 60 aa., 70 aa., 80 aa., 90 aa., 100 aa., . . . “Derivatives” may be recombinant or synthetic. The term “derivative” as used herein in relation to a protein means a chemically modified protein or an analogue thereof, wherein at least one substituent is not present in the unmodified protein or an analogue thereof, i.e. a protein which has been covalently modified. Typical modifications are amides, carbohydrates, alkyl groups, acyl groups, esters and the like. As used herein, the term “derivatives” also refers to longer or shorter polynucleotides/proteins and/or having e.g. a percentage of identity of at least 85%, 86%, 87%, 88%, 89%, 90%, 91%, 92%, 93%, 94%, 95%, 96%, 97%, 98%, more preferably of at least 99% with the sequences herein disclosed. In the present invention “at least 70% identity” means that the identity may be at least 70%, or 75%, or 80%, or 85% or 90% or 95% or 100% sequence identity to referred sequences. This applies to all the mentioned % of identity. Preferably, the % of identity relates to the full length of the referred sequence. The derivative of the invention also includes “functional mutants” of the polypeptides, which are polypeptides that may be generated by mutating one or more amino acids in their sequences and that maintain their activity. Indeed, the polypeptide of the invention, if required, can be modified in vitro and/or in vivo, for example by glycosylation, myristoylation, amidation, carboxylation or phosphorylation, and may be obtained, for example, by synthetic or recombinant techniques known in the art. In the present invention “functional” is intended for example as “maintaining their activity” e.g. immunomodulatory activity or anti-inflammatory activity. Also within the scope of the subject invention are polynucleotides which have the same nucleotide sequences of a polynucleotide exemplified herein except for nucleotide substitutions, additions, or deletions within the sequence of the polynucleotide, as long as these variant polynucleotides retain substantially the same relevant functional activity as the polynucleotides specifically exemplified herein (e.g., they encode a protein having the same amino acid sequence or the same functional activity as encoded by the exemplified or mentioned polynucleotide). Thus, the polynucleotides disclosed o referred herein should be understood to include mutants, derivatives, variants and fragments, as discussed above, of the specifically exemplified sequences. The subject invention also contemplates those polynucleotide molecules having sequences which are sufficiently homologous with the polynucleotide sequences of the invention so as to permit hybridization with that sequence under standard stringent conditions and standard methods (Maniatis, T. et al, 1982). Polynucleotides described herein can also be defined in terms of more particular identity and/or similarity ranges with those exemplified herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91 , 92, 93, 94, 95, 96, 97, 98, or 99% or greater as compared to a sequence exemplified herein. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

The peptide, the protein or the compounds as above defined may include an amino acid sequence with at least 65%, 70%, 75%, 80%, 82%, 85%, 90%, 92%, 95%, 98%, 99% or 100% identity to the sequences herein mentioned. Determining percent identity of two amino acid sequences may include aligning and comparing the amino acid residues at corresponding positions in the two sequences. If all positions in two sequences are occupied by identical amino acid residues then the sequences are said to be 100% identical. Percent identity may be measured by the Smith Waterman algorithm (Smith T F, Waterman M S 1981 “Identification of Common Molecular Subsequences,” J Mol Biol 147: 195-197, which is incorporated herein by reference as if fully set forth). The peptide, the protein or the compounds may have fewer or more than the residues of the mentioned sequences. The peptide, the protein or the compounds may present amino acid replacement in comparison to the sequence of the herein mentioned sequences. A sequence having less than 100% identity to the herein mentioned sequences may be referred to as a variant. In an embodiment, one or more amino acids residues are replaced with a residue having a crosslinking moiety.

The terms “variant” or “derivative” in relation to the amino acid sequences of the present invention includes any substitution of, variation of, modification of, replacement of, deletion of or addition of one (or more) amino acids from or to the sequence providing the resultant amino acid sequence preferably has targeting activity, preferably having at least 25 to 50% of the activity as the polypeptides herein presented, more preferably at least substantially the same activity. Thus, sequences may be modified for use in the present invention. Typically, modifications are made that maintain the activity of the sequence. Thus, in one embodiment, amino acid substitutions may be made, for example from 1, 2 or 3 to 10, 20 or 30 substitutions provided that the modified sequence retains at least about 25 to 50% of, or substantially the same activity. However, in an alternative embodiment, modifications to the amino acid sequences of a polypeptide of the invention may be made intentionally to reduce the biological activity of the polypeptide. For example, truncated polypeptides that remain capable of binding to target molecule but lack functional effector domains may be useful. In general, preferably less than 20%, 10% or 5% of the amino acid residues of a variant or derivative are altered as compared with the corresponding region.

Polypeptides of the invention also include fragments of the above-mentioned polypeptides and variants thereof, including fragments of the sequences. Preferred fragments include those which include an epitope. Suitable fragments will be at least about 5, e.g. 10, 12, 15 or 20 amino acids in length. They may also be less than 200, 100 or 50 amino acids in length. Polypeptide fragments of the proteins and allelic and species variants thereof may contain one or more (e.g. 2, 3, 5, or 10) substitutions, deletions or insertions, including conserved substitutions. Where substitutions, deletion and/or insertions have been made, for example by means of recombinant technology, preferably less than 20%, 10% or 5% of the amino acid residues are altered. Proteins of the invention are typically made by recombinant means. However, they may also be made by synthetic means using techniques well known to skilled persons such as solid phase synthesis.

The present invention is therefore illustrated by means of non-limiting examples in reference to the following figures.

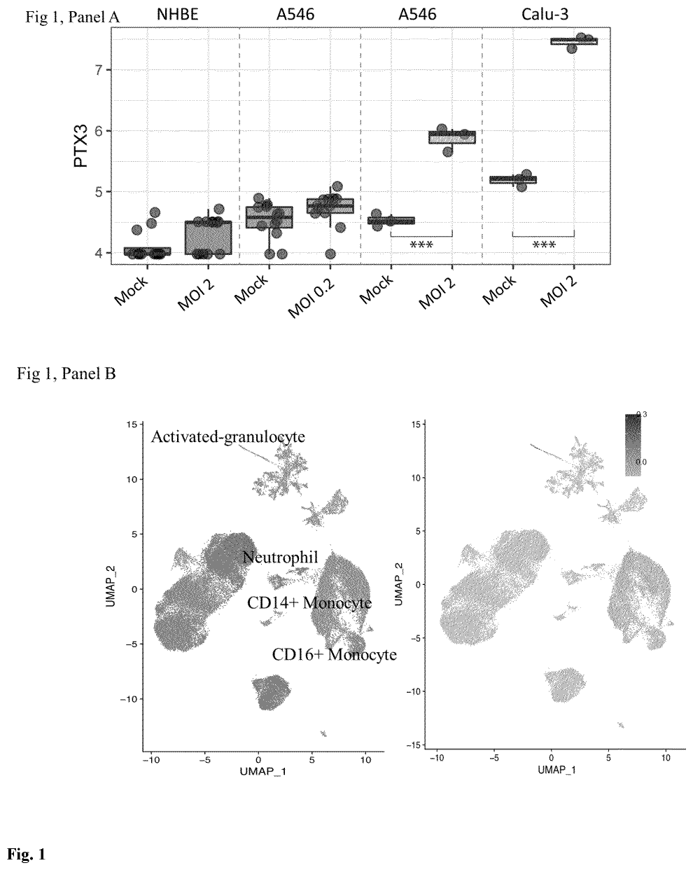

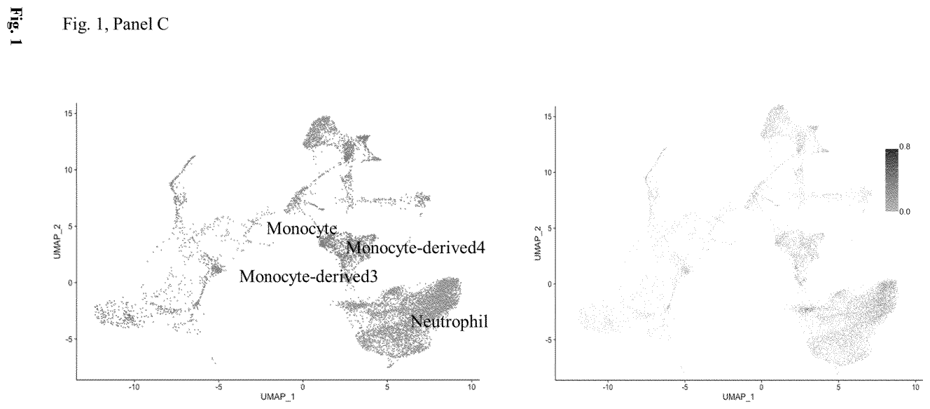

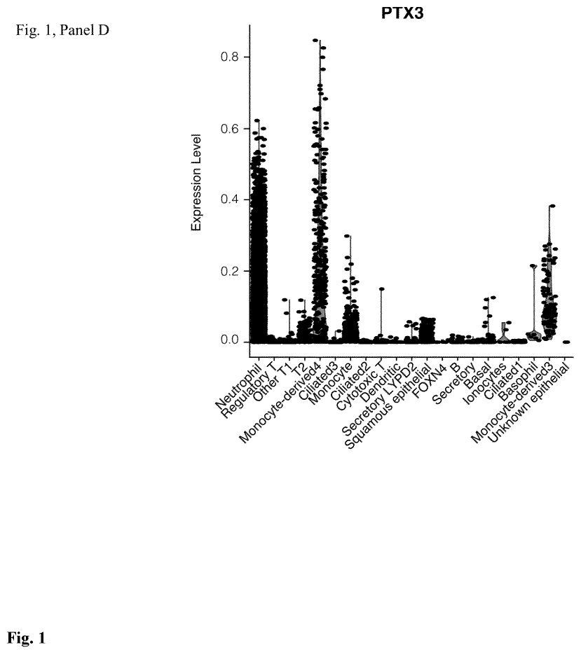

FIG. 1. In silico analysis of PTX3 expression in SARS-CoV-2 infected cells and COVID-19 patients. A) PTX3 expression in in vitro SARS-CoV-2 infected respiratory epithelial cells: normal human bronchial epithelial cells—NHBC (at MOI 2) and human lung cancer cell lines A549 (at MOI 0.2 and 2) and Calu-3 (at MOI 2). B) Single cell RNA-seq of COVID-19 PBMC. Right panel, Uniform Manifold Approximation and Projection (UMAP) map, showing Seurat-guided clustering of PBMC populations. Each point represents a single-cell colored according to cluster designation. Left panel: Heatmap showing PTX3 expression. C) Single cell RNA-seq of COVID-19 BALF. Upper panel, UMAP showing Seurat-guided clustering of BALF populations. Lower panel: Heatmap showing PTX3 expression. D) Violin plots representing expression and distribution of PTX3 within predicted BALF populations. Expression values are relieved after Rmagic imputation

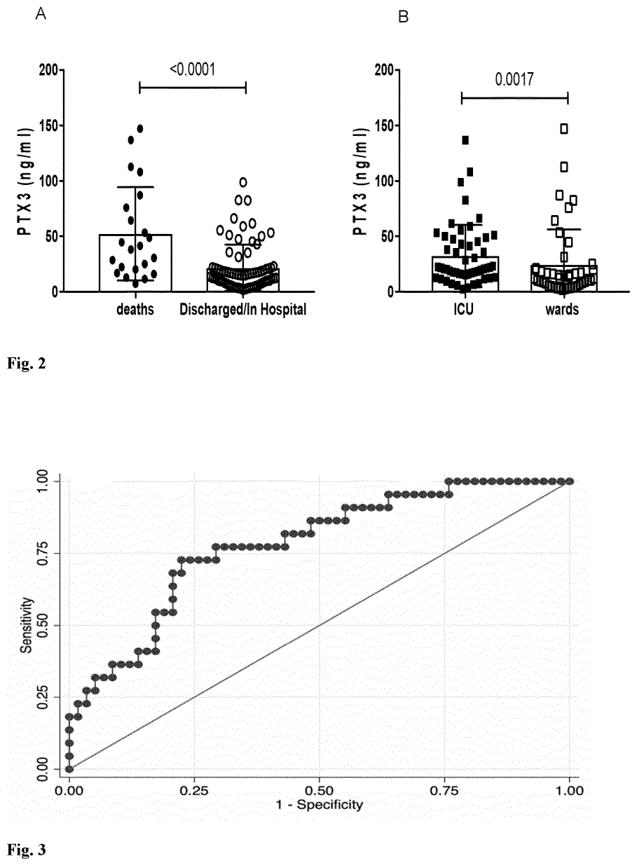

FIG. 2. PTX3 plasma levels in 96 COVID-19 patients admitted to Humanitas Clinical and Research Hospital.

Left panel (A): PTX3 plasma levels in patients based on the primary outcome (mortality). Right panel (B): PTX3 plasma levels in patients admitted to medical wards (50) or ICU (46).

FIG. 3. Receiver operating characteristic (ROC) curve for PTX3 for the primary outcome (death).

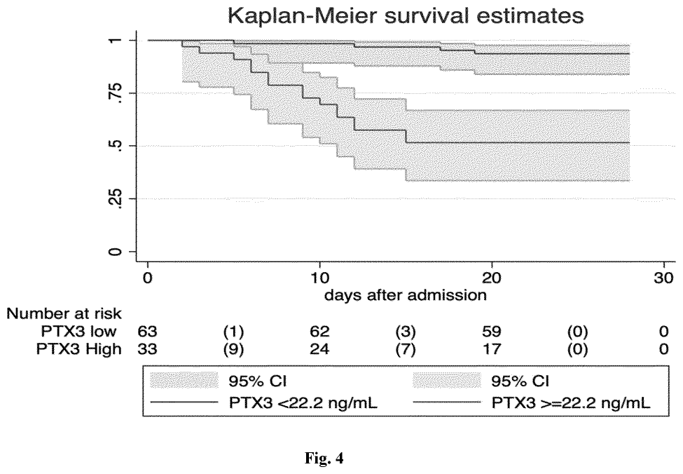

FIG. 4. Kaplan-Meier curves by level of PTX3. High level was defined as ≥of 22.2 ng/mL, low level as <22.2 ng/mL. The 28-day event-free survival was 0.94±0.03 (95% CI, 0.83 to 0.97) in low PTX3 group and 0.52±0.08 (95% CI, 0.34 to 0.67) in high PTX3 group. CI: confidence interval.

### EXAMPLES

### Methods

### Bioinformatic Analysis

Data relative to the transcriptional response to SARS-CoV-2 infection were derived from datasets deposited within the Gene Expression Omnibus (GEO). Data relative to bulk transcription in human normal bronchial (NHBE) and malignant cell lines (Calu-3 and A549) upon SARS-CoV-2 infection, were derived from the experiments within the series GSE147507²⁸. Raw bulk RNA-Seq reads were quality inspected with the software “FastQC” (https://www.bioinformatics.babraham.ac.uk/projects/fastqc/) and aligned with STAR (version 2.6.1)²⁹ on the GRCh38 genome guided by GENCODE annotation (version 33).

Gene summarized counts were processed in R, genes whose expression was major than 2 reads were filtered and vst normalized with the R package DESeq2^{30.} Significantly changing genes upon SARS-CoV-2 infection were obtained with DESeq2 by comparing each infected cell line with the respective mock-treated counterpart. Gene identifiers conversions were performed with the “org.Hs.eg.db” library (https://www.bioconductor.org/packages//2.10/data/annotation/html/org.Hs.eg.db.html). Plots were rendered with the R library “ggplot2” (https://ggplot2.tidyverse.org).

Available single cell RNA-Seq experiments, related to BALF of SARS-CoV2 individuals, were obtained from the public repositories Gene Expression Omnibus (GEO) and FigShare platform under the identifiers GSE145926, GSE149443 and the FigShare platform (https://figshare.com/articles/COVID-19_severity_correlates_with_airway_epithelium-immune_cell_interactions_identified_by_single-cell_analysis/12436517), scRNA-Seq datasets of SARS-CoV-2 infected PBMC (deposited in GEO under the series GSE150728)³¹ were explored with the portal cellxgene (https://chanzuckerberg.github.io/cellxgene/) and obtained from “The COVID19 Cell Atlas portal” (https://www.covid19cellatlas.org/#wilk20). Sparsecount matrices or Seurat objects were obtained as released and processed with the R package “Seurat”³² and confirmed with the published pipelines shared by respective authors.

Classification of clusters was performed according to the authors' parameters; the distribution of PTX3 expression was obtained after imputation with the “Rmagic” package³³.

### Study Design and Participants

This cohort study analyzed a cohort of 96 patients. Inventors included all males and non-pregnant females, 18 years of age or older, admitted to Humanitas Clinical and Research Center (Rozzano, Milan, Italy) between Mar. 4 and May 16, 2020 (data cutoff on May 13rd) with a laboratory-confirmed diagnosis of COVID-19. Hospital admission criteria were based on a positive assay for SARS-CoV-2 associated with respiratory failure requiring oxygen therapy, or radiological evidence of significant pulmonary infiltrates on chest computed tomography (CT) scan, or reduction in respiratory/cardiopulmonary reserve as assessed by 6 minutes walking test, or due to frailty related with patient comorbidity. Inventors assessed an outcome of death. 52 patients of 96 (54%) were transferred to ICU because requiring invasive ventilation or non-invasive mechanical ventilation with oxygen fraction over 60%. Patients with continuous positive airway pressure therapy (CPAP) were followed up by ICU outreach team and ward physicians in COVID-19 wards. Acute respiratory syndrome (ARDS) was defined according to the Berlin definition³⁴.

### Laboratory Test, Demographic, and Medical History

Laboratory testing at hospital admission included: complete blood count, renal and liver function (transaminase, total/direct/indirect bilirubin, gamma-glutamyl transferase, alkaline phosphatase), creatinine kinase, lactate dehydrogenase, myocardial enzymes, electrolytes and triglycerides. A panel of acute phase reactants including interleukin-6 (IL-6), serum ferritin, D-dimer, C-reactive protein (CRP), fibrinogen, and procalcitonin (PCT) was performed. Body temperature, blood pressure, heart rate, peripheral saturation, and respiratory rate were measured in all patients. Chest CT scan and arterial blood gas analysis were performed in the emergency department. In all patients PTX3 was measured within the first few days after the admission date (mean 2.1+1.6 days). Pneumococcal and Legionella urinary antigen tests were routinely performed. Nasopharyngeal swab for influenza A, B and H1N1 was also routinely performed to exclude co-infections. Additional microbiological tests were performed to exclude other pathogens as possible etiological agents when suggested by clinical conditions (bacterial cultures of sputum, blood and urine). Inventors obtained a comprehensive present and past medical history from patients. Positivity was assessed on the basis of reverse-transcriptase-polymerase-chain-reaction (RT-PCR) assay for SARS-CoV-2 on a respiratory tract sample tested by our laboratory, in accordance with the protocol established by the WHO (https://www.who.int/emergencies/diseases/novel-coronavirus-2019/technical-guidance/laboratory-guidance). Due to the high false negative rate of RT-PCR from pharyngeal swab, two different swabs were performed in every patient to increase the detection rate³⁵. In cases of negative assay in throat-swab specimens, but with suggestive clinical manifestations, presence of contact history or suggestive radiological evidence for COVID-19, the detection was performed on bronchoalveolar lavage fluid (BAL) or endotracheal aspirate, which has higher diagnostic accuracy. All demographics, medical history and laboratory tests were extracted from electronic medical records and were checked by a team of three expert physicians. The study was approved by the local Ethical Committee (authorization 233/20), and the requirement for informed consent was waived.

### Sample Collection and PTX3 Measurement

Venous blood samples were collected during the first 5 days after hospital admission (mean 2.1±standard deviation 1.6 days), centrifuged, and EDTA plasma was stored at −80° C. until use. PTX3 plasma levels were measured, as previously described¹⁸, by a sandwich ELISA (detection limit 0.1 ng/mL, inter-assay variability from 8 to 10%) developed in-house, by personnel blind to patients' characteristics. In each analytical session a sample obtained from a pool of plasma from healthy donors was used as internal control. The mean PTX3 concentration measured in this sample was 1.88±0.6 ng/mL.

### Statistical Methods

Demographic, clinical, laboratory, and outcome data were obtained from electronic medical records and patient chart notes using a standardized data collection form. Descriptive statistics included means with standard deviations (SD) and medians with interquartile ranges (IQR) for continuous variables, and frequency analyses (percentages) for categorical variables. Linearity of continuous variables was checked by comparing models with the linear term to the model with restricted cubic splines. The optimal cut-off levels of PTX3 for predicting 28-day outcome of death have been investigated using receiver operating characteristic (ROC) curves. The correlation between variables and sequential organ failure assessment (SOFA) score was evaluated by Spearman's rank correlation coefficient (rho). To identify the association between PTX3 levels and the outcome in hospitalized COVID-19 patients, inventors used time-to-event (survival) methods for censored observations. The composite study endpoint was “death” within 28 days from hospital admission. Time to event was defined as the time from hospital admission until the date of event or censoring. Patients discharged early and alive from the hospital, and without having experienced ICU transfer, were considered event-free through day 28³⁶. May 13, 2020 was considered as data cutoff. Kaplan-Meier estimates were used to draw the cumulative incidence curves, compared by log-rank tests. Furthermore, multivariable Cox proportional hazards (PH) models of prognostic factors were used. The analyses were based on non-missing data (missing data not imputed). Confounders were selected according to a review of the literature, statistical relevance, and consensus opinion by an expert group of physicians and methodologists. After fitting the model, the PH assumption was examined on the basis of Schoenfeld residuals. The hazard ratios (HR) were presented with their 95% confidence intervals (CI) and the respective p-values. A ratio higher than 1.0 implies a higher probability of event compared to the reference group.

### Results

### PTX3 Expression is Induced in COVID-19

Inventors conducted an in silico bioinformatic analysis of the expression of PTX3 using public databases. As shown in FIG. 1A SARS-CoV-2 strongly induced or amplified PTX3 transcript expression in three lines representative of respiratory tract epithelial cells (Calu-3; A549; human normal bronchial cells, NHBE) (dataset GSE147507)²⁸. Bioinformatic analysis at single cell level of peripheral blood monononuclear cells obtained from COVID-19 patients, revealed that PTX3 was selectively expressed by COVID-19 monocytes (dataset GSE150728)³¹ (FIG. 1B). Interestingly, at single cell level CD16 monocytes were negative for PTX3 expression (not shown). Moreover, bioinformatic analysis at single cell level of COVID-19 bronchoalveolar lavage cells (https://www.medrxiv.org/content/10.1101/2020.04.29.20084327)³⁷ revealed that COVID-19 was strongly expressed in neutrophils and monocyte-macrophage populations, as identified by molecular signatures (FIG. 1C, 1D). Epithelial cells in the fluid were negative.

### Prognostic Significance of PTX3

Demographic, clinical, and laboratory features of patients were shown in Table 1. All variables were also stratified by clinical outcome death versus alive (Table 1).

In the overall population, patients had elevated PTX3 (mean 28.7±29.8 ng/mL; median 17.3 ng/mL, IQT 10.0-39.8 ng/mL), CRP (mean 14.2±10.3 mg/dL; median 12.29 mg/dL, IQT 6.22-22.0 mg/dL), and IL-6 levels (median 48 pg/ml; IQT 21-115 pg/ml). Similarly to our previous study³⁸, inventors also observed elevated levels of ferritin (mean 634±608 μg/mL; median 536 μg/mL, IQT 151-822 μg/mL), D-dimer (mean 2404±7448 μg/mL; median 741 μg/mL, IQT 476-1456 μg/mL), and LDH (mean 371±168 U/L; median 345 U/L, IQT 268-424 U/L). A reduction in lymphocytes (mean 0.86±0.43×10³/mL; median 0.8×10³/mL, IQT 0.55-1.1×10³/mL) and eosinophils (mean 0.06±0.111×10³/mL; median 0×10³/mL, IQT 0-0.1×10³/mL) were also found, as previously reported³⁸.

In our cohort, 52 patients (54.2%) were transferred to the ICU within 7 days from admission because of clinical worsening. The primary endpoint of death occurred in 22 patients (23%), comprised of 14 patients who died in ICU and 8 in medical wards, while a total of 58 patients (60.4%) had been discharged and 16 (16.6%) were still hospitalized. PTX3 values were higher in dead patients compared to survivors (mean 52.3±41.8 ng/mL; median 39.8 ng/mL, IQT 20.2-75.7 ng/mL versus mean 21.0±20.7 ng/mL; median 15.3 ng/mL, IQT 8.2-21.3 ng/mL, respectively; Table 1 and FIG. 2 panel A); moreover, higher PTX3 levels were also found in ICU patients compared to ward patients (mean 32.4±27.7 ng/mL; median 21.0 ng/mL, IQT 15.6-46.3 ng/mL versus mean 24.3±31.8 ng/mL; median 12.4 ng/mL, IQT 6.12-20.2 ng/mL, respectively; FIG. 2 panel B).

The best AUC value for the prediction of death at 28 days was calculated for PTX3 (0.75, 95% CI 0.64-0.86). The most valid cut-off level to predict 28-day mortality was PTX3=22.25 ng/mL (sensitivity 0.73; specificity 0.78; FIG. 3). The Kaplan-Meier curve showed an overall 28-day event-free survival of 0.94±0.03 (95% CI, 0.83 to 0.97) in low PTX3 group (<22.25 ng/mL) and 0.52±0.09 (95% CI, 0.34 to 0.67) in high PTX3 group (≥22.25 ng/mL; Log-ranks test p<0.0001; FIG. 4). In the univariate COX analysis, PTX3 was a strong predictor of mortality in COVID-19 patients (unadjusted HR for ≥22.25 versus <22.25 ng/mL, 10.25; 95% CI, 3.41 to 30.75; p<0.0001). Basing on available literature and statistical relevance, inventors adjusted the model for possible confounding factors (age, ICU stay, and SOFA score at admission). PTX3 was confirmed as a strong predictor of short-term mortality (adjusted HR for ≥22.25 vs <22.25 ng/mL, 7.8; 95% CI, 2.5 to 24.0; p<0.0001; Table 2). PH assumption was not violated (global test of PH assumption, p=0.153).

Correlations between PTX3 and other inflammatory markers were initially assessed with Spearman test and were reported in Table 6. PTX3 was significantly correlated to CRP, procalcitonin, IL-6, ferritin, and D-dimer, but also with other COVID-19 poor prognostic factors, such as LDH, troponin-I, lymphocyte count (Table 1s). PTX3, CRP, ferritin, IL-6, and D-dimer were also analyzed as continuous values for predicting mortality (univariate Cox regression; Table 3). PTX3 was confirmed significantly associated to mortality; moreover, IL-6 and D-dimer were also significantly, but weakly, associated with mortality at the unadjusted analysis. Performing a multivariate logistic regression including all these markers (PTX3, ferritin, D-Dimer, IL-6, and CRP), PTX3 was the only inflammatory marker significantly associated with death (adjusted HR for 1 ng/mL increase, 1.13; 95% CI, 1.02 to 1.24; p=0.021; Table 4).

To investigate if PTX3 could be also considered a biomarker of COVID-19 severity at the time of hospital admission, identified by basal SOFA score ≥3, inventors performed a multivariate logistic regression including PTX3, CRP, IL-6, and D-dimer. CRP was the only factor significantly associated to SOFA score ≥3 (Table 5).

### DISCUSSION

As of May 25 2020 the case fatality rate of COVID-19 in Italy is reported to be 14.3% with an average ICU admission rate of more than 20.4%³⁹. Elevated levels of CRP, cytokines, and chemokines^{8.38,40} together with low lymphocyte and eosinophil counts characterize patients with severe disease⁴¹. However, a reliable biomarker of poor outcome in COVID-19 is still lacking. A delayed identification of severe cases might lead to delayed intensive care treatment and increased mortality. On the contrary, the early and accurate triaging of the patients may contribute to a timely and rationale planning of ICU admissions. The present study was designed to investigate expression and clinical significance of the fluid phase pattern recognition molecule PTX3 in COVID-19. Inventors found that PTX3 was induced by SARS-CoV-2 in respiratory tract epithelial cells. In COVID-19 patients, PTX3 analyzed at single cell level was selectively expressed by monocytes among circulating cells and by lung macrophages. High PTX3 plasma levels (≥22.25 ng/mL) were a strong independent indicator of short term 28-day mortality with an adjusted Hazard Ratio of 7.8 (95% CI 2.5-24). In this patient cohort, PTX3 fared definitely better than other known prognostic markers including CRP, IL-6, ferritin and D-dimer.

PTX3 serum levels above the normal value (<2 ng/mL⁴²) can be found in the subclinical inflammatory status of cardiovascular disease 43 as well as in infections and sepsis, with increasing median values when moving to more severe conditions. In a large study conducted in 1326 unselected hospitalized subjects (14% with infectious diseases), PTX3 above 95th percentile of healthy non-hospitalized subjects (>6.4 ng/mL) was significantly associated to higher mortality in the short term, independently of hospitalization causes (adjusted HR 5, 95%CI 2.9-8.8)⁴⁴. Elevated PTX3 serum levels are indeed not related to a specific diagnosis rather predict severe cases or poor prognosis in different contexts characterized by a systemic inflammatory response⁴⁴. In a recent, prospective, observational study including 547 ICU patients (42.4% with infections), a PTX3 cut off similar to that identified in our study was reported to predict mortality: PTX3 serum level above the median cohort value of 20.9 ng/mL was independently associated to 28-day mortality when adjusted for age, sex, chronic diseases, and immunosuppression (HR 1.87, 95% CI 1.41-2.48)⁴⁵. In another recent paper conducted on 281 sepsis patients, serum PTX3>26 ng/mL was associated to mortality⁴⁶. Taken together, these findings and our results suggest that circulating PTX3 levels ten-fold above the normal value reflect a severe systemic inflammatory involvement with ominous outcome.

PTX3 has been shown to be produced by diverse cell types including myelomonocytic cells, lung epithelial cells and endothelial cells. In the present study, inventors found that SARS-CoV-2 induced gene expression of PTX3 in respiratory tract epithelial cells. Peripheral blood mononuclear cells represent the easily accessible cellular source in patients. By bioinformatic analysis at single cell level, inventors found that PTX3 was selectively expressed by monocytes among circulating leukocytes. Moreover, in lung bronchoalveolar lavage fluid, single cell analysis revealed selective expression of PTX3 in neutrophils and macrophages, which play a major role in the pathogenesis of the disease^{3,4}.

The PTX3 gene was originally cloned in endothelial cells¹¹ and vascular cells are a major source of this component of humoral innate immunity, though their role could not be directly ascertained in the present study. Endothelial cells and the lung vascular bed have emerged as major determinant of COVID-19-associated microvascular thrombosis and disease pathogenesis⁹. PTX3 plasma levels have been shown to correlate with severity of disease in various forms of vascular pathology including small vessel vasculitis, coronary heart disease, and Kawasaki disease^{27,43,47}. The latter observation raises the issue of its significance in the Kawasaki-like disease observed in children after COVID-19 (e.g.^{48,49}). Of interest, our data show a significant correlation between PTX3 and D-dimer, surrogate of coagulation cascade activation and marker of venous thrombosis, and between PTX3 and troponin-I, marker of myocardial disease: both myocardial inflammation and acute ischemic heart disease have been described in COVID-19^{50,51}. These observations raise the possibility that the strong prognostic significance of PTX3 in COVID-19 may reflect its positioning at the very intersection between macrophage-driven inflammation and vascular involvement.

In conclusion, the results presented here suggest that high PTX3 plasma levels (≥22 ng/mL) are strongly associated with unfavorable COVID-19 disease progression, defined as 28-day mortality, and may serve as a useful prognostic biomarker to decide intensity of care based on the predicted individual risk of death. PTX3 fared better than other classic biomarkers including CRP and IL-6. Given the relatively small sample size (96 patients) this finding should be interpreted with caution. With this caveat, it is tempting to speculate that PTX3 plasma levels may better reflect local tissue disruptive inflammation including the involvement of myelomonocytic cells and the vascular bed. The significance of PTX3 as a biomarker in COVID-19 patient management and stratification and its role in the virus-host interaction deserve further studies.

### TABLES

TABLE 1
Demographics, laboratory and clinical characteristics of COVID-19
hospitalized patients grouped by different outcome.

| | Patients | Alive patients | Death patients | |
|---|---|---|---|---|
| Variables | (n = 96) | (n = 74) | (n = 22) | |
| Demographic Characteristics | Mean ± SD; Median (IQR) or n (%) | | | |
| Age (years)* | 65.2 ± 15.2; | 62.5 ± 15.1; | 74.6 ± 11.3; | |
| | 65.1 (56-74.5) | 61 (51-73) | 73 (69-83) | |
| Gender | | | | |
| Female | 31 (33) | 25 (34) | 6 | (27) |
| Male | 65 (67) | 49 (66) | 16 | (73) |

Laboratory Characteristics
Blood Biochemistry

| | | | |
|---|---|---|---|
| White blood cells (×10⁹/L; | 9.2 ± 5.6; | 9.3 ± 6; | 8.9 ± 4.1; |
| normal range 4.0-10.0) | 7.7 (5.9-10.4) | 7.5 (5.8-10.9) | 8 (6.6-9.7) |
| Neutrophils (×10⁹/L; | 7.7 ± 5.4; | 7.7 ± 5.8; | 7.6 ± 4; |
| normal range 2.0-7.0) | 6.2 (4.4-8.9) | 6 (4.3-9) | 7 (5-8.4) |
| Lymphocytes (×10⁹/L; | 0.9 ± 0.4; | 0.9 ± 0.4; | 0.7 ± 0.4; |
| normal range 1.0-4.0)* | 0.8 (0.6-1.1) | 0.9 (0.6-1.2) | 0.6 (0.4-0.8) |
| Eosinophils (×10⁹/L; | 0.1 ± 0.1; | 0.1 ± 0.1; | 0.1 ± 0.1; |
| normal range 1.0-5.0) | 0.0 (0.0-0.1) | 0.0 (0.0-0.1) | 0.1 (0.0-0.2) |
| Hemoglobin (g/dL; | 12.5 ± 1.9; | 12.5 ± 1.9; | 12.5 ± 1.8; |
| normal range 13.0-16.0) | 12.6 (11.3-13.7) | 12.7 (11.3-13.6) | 12.4 (11.6-13.9) |
| Platelets (×10⁹/L; | 253 ± 105; | 271 ± 100; | 195 ± 100; |
| normal range 150-400)** | 238 (172-313) | 247 (198-332) | 156 (100-237) |
| Alanine aminotransferase | 38 ± 37; | 39 ± 38; | 33 ± 21; |
| (U/L; normal range <51) | 26 (20-42) | 26 (20-43) | 28 (19.5-39) |
| Aspartate aminotransferase | 50 ± 56.5; | 49 ± 60; | 56 ± 38; |
| (U/L; normal range <51) | 32 (22-49) | 32 (21-42) | 51 (32.5-60.5) |
| Gamma-glutamyl | 52 ± 57; | 48 ± 38; | 70 ± 122; |
| tanspeptidase (U/L; | 28 (18-74) | 32 (18-76) | 27 (18-41) |
| normal range <55) | | | |
| Alkaline phosphatase (U/L; | 108 ± 54; | 105 ± 55; | 122 ± 50; |
| normal range 40-150) | 90 (75-124) | 89 (74-111) | 121 (78-184) |
| Total bilirubin (mg/dL; | 0.9 ± 0.6; | 0.9 ± 0.6; | 1.1 ± 0.6; |
| normal range 0.3-1.2) | 0.8 (0.6-1.2) | 0.7 (0.5-1.2) | 0.9 (0.7-1.3) |
| Direct bilirubin (mg/dL; | 0.2 ± 0.2; | 0.2 ± 0.1; | 0.4 ± 0.3; |
| normal range <0.3)** | 0.2 (0.1-0.3) | 0.2 (0.1-0.2) | 0.4 (0.2-0.5) |
| Indirect bilirubin (mg/dL; | 0.6 ± 0.2; | 0.5 ± 0.2; | 0.9 ± 0.3; |
| normal range 0.05-1.10)** | 0.6 (0.4-0.7) | 0.5 (0.4-0.6) | 0.8 (0.7-0.9) |
| Creatine kinase (U/L; | 183 ± 222; | 151 ± 177; | 348 ± 347; |
| normal range <172) | 105 (58-184) | 98 (53-180) | 150 (92-651) |
| Lactate dehydrogenase (U/L; | 372 ± 169; | 338 ± 146; | 483 + 196; |
| normal range <248)* | 345 (268-424) [n = 61] | 326 (240-382) [n = 47] | 412 (380-505) [n = 14] |
| Serum creatinine (mg/dL; | 1.2 ± 1.2; | 1.2 ± 1.3; | 1.2 ± 0.7; |
| normal range 0.67-1.17) | 0.9 (0.7-1.2) | 1.0 (0.7-1.7) | 0.9 (0.8-1.5) |
| Troponin-I (ng/L; | 219.5 ± 112.6; | 25.2 ± 34.2; | 1215.4 ± 2669.6; |
| normal range 1-35)** | 12.3 (6.3-46.6) [n = 49] | 11.1 (5.7-26.9) [n = 41] | 93.9 (35.6-865.5) [n = 8] |
| D-dimer (μg/mL; | 2136 ± 6583; | 1521 ± 2668; | 5083 ± 14795; |
| normal range 0.2-0.35) | 637 (409-1394) [n = 87] | 625 (366-1376) [n = 72] | 720 (509-2735) [n = 15] |
| Fibrinogen (mg/dL; | 507 ± 169; | 516 ± 158; | 462 ± 225.6; |
| normal range 160-400) | 488 (410-586) | 481 (410-586) | 510 (344-558) |
| Triglycerides (mg/dL; | 150 ± 78; | 141 ± 60; | 198 ± 136; |
| normal range 10-150) | 134 (105-171) | 133 (107-161) | 164 (89-279) |

Infection-Related Biomarkers

| | | | |
|---|---|---|---|
| Pentraxin 3 (ng/mL)** | 28.7 ± 29.8; | 21 ± 20.7; | 52.3 ± 41.8; |
| | 17.3 (10-39.8) | 15.3 (8.2-21.3) | 39.8 (20.2-75.7) |
| Interleukin-6 (pg/mL; | 115 ± 247; | 85 ± 141; | 303 ± 564; |
| normal range <6.4) | 56 (21-115) [n = 51] | 41 (19-103) [n = 44] | 82 (42-228) [n = 7] |
| C-reactive protein (mg/dL; | 14.2 ± 10.4; | 13.3 ± 10; | 17.7 ± 11.2; |
| normal range <1.0) | 12.3 (6.2-22) | 12.2 (4.8-20.2) | 15 (6.6-26.4) |
| Procalcitonin (ng/mL; | 1.7 ± 7; | 1.9 ± 7.9; | 1.1 ± 1.6; |
| normal range 0.05-0.5) | 0.4 (0.1-1.4) [n = 91] | 0.3 (0.1-1.4) [n = 70] | 0.6 (0.4-0.8) [n = 21] |
| Ferritin (ng/mL; | 760 ± 721; | 693 ± 675; | 1133 ± 883; |
| normal range 23.9-336.2) | 622 (185-976) [n = 66] | 567 (173-881) [n = 56] | 867 (522-1624) [n = 10] |

Severity-Related Biomarkers

| | | | |
|---|---|---|---|
| Respiratory rate (breaths | 19 ± 3; | 18 ± 2; | 21 ± 5; |
| per minute) | 18 (18-19.5) | 18 (17-19) | 19 (18-20) |
| Ratio of PaO2 to FiO2* | 259 ± 140; | 277 ± 144; | 201 ± 110; |
| | 200 (149-384) [n = 88] | 230 (160-393) [n = 67] | 142 (130-233) [n = 21] |
| Pulse (beats per minute) | 86 ± 15; | 84 ± 15; | 96 ± 13; |
| | 85 (76-95) | 84 (72-90) | 95 (90-101)* |
| Mean pressure (mmHg) | 86 ± 14; | 87 ± 15; | 83 ± 10; |
| | 86 (74-94) [n = 92] | 87 (72-111) [n = 70] | 84 (74-89) |
| Temperature (° C.) | 36.9 ± 0.8; | 36.9 ± 0.8; | 36.8 ± 0.9; |
| | 36.5 (36.2-37.5) | 36.7 (36.2-37.5) | 36.4 (36.2-38) |
| SOFA* | 4 ± 3; | 3.5 ± 2.8; | 5.6 ± 2.7; |
| | 4 (1-6) | 3 (1-6) | 6 (4-7) |

Comorbidities

| Hypertension | 50 (52) | 39 (53) | 11 | (50) |
|---|---|---|---|---|
| Chronic heart diseases | 14 (15) | 11 (15) | 3 | (14) |
| Atrial fibrillation | 12 (13) | 10 (14) | 2 | (9) |
| Diabetes type 2 | 29 (30) | 22 (30) | 7 | (32) |
| Obesity (BMI) | 28.3 ± 7.7; | 28.3 ± 8.3; | 27.8 ± 4.0; | |
| | 26 (23-31) [n = 80] | 26 (23-33) [n = 66] | 27.7 (24-29) [n = 14] | |
| Chronic obstructive | 9 (9) | 7 (9) | 2 | (9) |
| pulmonary disease | | | | |
| Chronic Kidney disease | 8 (8) | 6 (8) | 2 | (9) |
| History of neoplasia | 14 (15) | 14 (19) | 0 | (0) |
| Stroke | 9 (9) | 5 (7) | 4 | (18) |

Mann-Whitney test between death and alive patients:
*p < 0.05;
**p < 0.001

TABLE 2
PTX3 as predictor of death in Hospitalized COVID-19 patients
(multivariate Cox model, HR adjusted for confounders:
age, stay in ICU, and SOFA score at admission).

| Variables | HR | Std. Error | 95% CI | p-Value |
|---|---|---|---|---|
| PTX3 (≥22.25 ng/mL) | 7.8 | 4.47 | (2.54-24.03) | <0.0001 |
| Age | 1.09 | 0.036 | (1.03-1.17) | 0.005 |
| ICU | 1.76 | 1.43 | (0.36-8.61) | 0.485 |
| SOFA | 1.22 | 0.12 | (1.01-1.47) | 0.036 |

TABLE 3
Inflammatory and other biomarkers as predictors of death in
Hospitalized COVID-19 patients (univariate Cox models).

| Variables | HR | Std. Error | 95% CI | p-Value |
|---|---|---|---|---|
| PTX3 | 1.031 | 0.006 | (1.019-1.042) | <0.001 |
| C-reactive protein | 1.036 | 0.021 | (0.995-1.078) | 0.081 |
| Interleukin-6 | 1.001 | 0.001 | (1.001-1.003) | 0.01 |
| Procalcitonin | 0.979 | 0.056 | (0.875-1.096) | 0.72 |
| Ferritin | 1.0005 | 0.0003 | (0.999-1.001) | 0.09 |
| D-dimer | 1.0001 | 0.00003 | (1.00003-1.0002) | 0.005 |

TABLE 4
Correlations between inflammatory and other biomarkers
levels (at admission) and outcome (death) in Hospitalized
COVID-19 patients (multivariate logistic regression).

| Variables | OR | Std. Error | 95% CI | p-Value |
|---|---|---|---|---|
| PTX3 | 1.13 | 0.06 | (1.02-1.24) | 0.021 |
| C-reactive protein | 0.90 | 0.11 | (0.70-1.14) | 0.38 |
| Interleukin 6 | 1.0004 | 0.0032 | (0.9941-1.0066) | 0.908 |
| Ferritin | 1.0004 | 0.0010 | (0.9983-1.0024) | 0.709 |
| D-Dimer | 0.999 | 0.001 | (0.998-1.001) | 0.912 |

TABLE 5
Correlations between inflammatory and other biomarkers levels
and systemic organ failure (SOFA score ≥3) at admission in
Hospitalized COVID-19 patients (multivariate logistic regression).

| Variables | OR | Std. Error | 95% CI | p-Value |
|---|---|---|---|---|
| PTX3 | 1.01 | 0.0128 | (0.98-1.03) | 0.508 |
| C-reactive protein | 1.14 | 0.0481 | (1.05-1.23) | 0.003 |
| Interleukin 6 | 1.00 | 0.0009 | (0.998-1.002) | 0.927 |
| D-Dimer | 1.00 | 0.0003 | (1.0-1.001) | 0.218 |

TABLE 6
Spearman correlation between PTX3 and other
inflammatory or biochemical markers.

| Variables | ρ | p-Value |
|---|---|---|
| Neutrophils | 0.0929 | 0.368 |
| Lymphocytes | −0.3048 | 0.002 |
| C-reactive protein | 0.3156 | 0.002 |
| Procalcitonin | 0.2841 | 0.006 |
| Interleukin-6 | 0.5353 | <0.001 |
| Ferritin | 0.4604 | <0.001 |
| D-dimer | 0.2945 | 0.017 |
| Lactate dehydrogenase | 0.5443 | <0.001 |
| Platelets | 0.0297 | 0.808 |
| Troponin-I | 0.5552 | <0.001 |

### REFERENCES

1. Wu F, Zhao S, Yu B, et al. A new coronavirus associated with human respiratory disease in China. Nature 2020; 579(7798): 265-9.
2. Zhu N, Zhang D, Wang W, et al. A Novel Coronavirus from Patients with Pneumonia in China, 2019. N Engl J Med 2020; 382(8): 727-33.
3. Fauci A S, Lane H C, Redfield R R. Covid-19—Navigating the Uncharted. N Engl J Med 2020; 382(13): 1268-9.
4. Cecconi M, Forni G, Mantovani A. Ten things we learned about COVID-19. Intensive Care Med 2020: 10.1007/s00134-020-6140-0.
5. Chen N, Zhou M, Dong X, et al. Epidemiological and clinical characteristics of 99 cases of 2019 novel coronavirus pneumonia in Wuhan, China: a descriptive study. Lancet 2020; 395(10223): 507-13.
6. Wolfel R, Corman V M, Guggemos W, et al. Virological assessment of hospitalized patients with COVID-2019. Nature 2020; 581(7809): 465-9.
7. Xu Z, Shi L, Wang Y, et al. Pathological findings of COVID-19 associated with acute respiratory distress syndrome. Lancet Respir Med 2020; 8(4): 420-2.
8. Huang C, Wang Y, Li X, et al. Clinical features of patients infected with 2019 novel coronavirus in Wuhan, China. Lancet 2020; 395(10223): 497-506.
9. Ackermann M, Verleden S E, Kuehnel M, et al. Pulmonary Vascular Endothelialitis, Thrombosis, and Angiogenesis in Covid-19. N Engl J Med 2020: 10.1056/NEJMoa2015432.
10. Varga Z, Flammer A J, Steiger P, et al. Endothelial cell infection and endotheliitis in COVID-19. Lancet 2020; 395(10234): 1417-8.
11. Garlanda C, Bottazzi B, Magrini E, Inforzato A, Mantovani A. PTX3, a Humoral Pattern Recognition Molecule, in Innate Immunity, Tissue Repair, and Cancer. Physiol Rev 2018; 98(2): 623-39.
12. Cunha C, Aversa F, Lacerda J F, et al. Genetic PTX3 deficiency and aspergillosis in stem-cell transplantation. The New England journal of medicine 2014; 370(5): 421-32.
13. Mairuhu A T, Peri G, Setiati T E, et al. Elevated plasma levels of the long pentraxin, pentraxin 3, in severe dengue virus infections. J Med Virol 2005; 76(4): 547-52.
14. Sprong T, Peri G, Neeleman C, et al. Pentraxin 3 and C-reactive protein in severe meningococcal disease. Shock 2009; 31(1): 28-32.
15. Wagenaar J F, Goris M G, Gasem M H, et al. Long pentraxin PTX3 is associated with mortality and disease severity in severe Leptospirosis. J Infect 2009; 58(6): 425-32.
16. Caironi P, Masson S, Mauri T, et al. Pentraxin 3 in patients with severe sepsis or shock: the ALBIOS trial. Eur J Clin Invest 2017; 47(1): 73-83.
17. Muller B, Peri G, Doni A, et al. Circulating levels of the long pentraxin PTX3 correlate with severity of infection in critically ill patients. Crit Care Med 2001; 29(7): 1404-7.
18. Mauri T, Bellani G, Patroniti N, et al. Persisting high levels of plasma pentraxin 3 over the first days after severe sepsis and septic shock onset are associated with mortality. Intensive Care Med 2010; 36(4): 621-9.
19. Latini R, Maggioni A P, Peri G, et al. Prognostic significance of the long pentraxin PTX3 in acute myocardial infarction. Circulation 2004; 110(16): 2349-54.
20. Jenny N S, Arnold A M, Kuller L H, Tracy R P, Psaty B M. Associations of pentraxin 3 with cardiovascular disease and all-cause death: the Cardiovascular Health Study. Arterioscler Thromb Vasc Biol 2009; 29(4): 594-9.
21. Latini R, Gullestad L, Masson S, et al. Pentraxin-3 in chronic heart failure: the CORONA and GISSI-HF trials. Eur J Heart Fail 2012; 14(9): 992-9.
22. Kotooka N, Inoue T, Fujimatsu D, et al. Pentraxin3 is a novel marker for stent-induced inflammation and neointimal thickening. Atherosclerosis 2007.
23. Knoflach M, Kiechl S, Mantovani A, et al. Pentraxin-3 as a marker of advanced atherosclerosis results from the Bruneck, ARMY and ARFY Studies. PLoS One 2012; 7(2): e31474.
24. Fazzini F, Peri G, Doni A, et al. PTX3 in small-vessel vasculitides: an independent indicator of disease activity produced at sites of inflammation. Arthritis Rheum 2001; 44(12): 2841-50.
25. van Rossum A P, Pas H H, Fazzini F, et al. Abundance of the long pentraxin PTX3 at sites of leukocytoclastic lesions in patients with small-vessel vasculitis. Arthritis Rheum 2006; 54(3): 986-91.
26. Jenny N S, Blumenthal R S, Kronmal R A, Rotter J I, Siscovick D S, Psaty B M. Associations of pentraxin 3 with cardiovascular disease: the Multi-Ethnic Study of Atherosclerosis. J Thromb Haemost 2014; 12(6): 999-1005.
27. Ramirez G A, Rovere-Querini P, Blasi M, et al. PTX3 Intercepts Vascular Inflammation in Systemic Immune-Mediated Diseases. Front Immunol 2019; 10: 1135.
28. Blanco-Melo D, Nilsson-Payant B E, Liu WC, et al. Imbalanced Host Response to SARS-CoV-2 Drives Development of COVID-19. Cell 2020; 181(5): 1036-45 e9.
29. Dobin A, Davis C A, Schlesinger F, et al. STAR: ultrafast universal RNA-seq aligner. Bioinformatics 2013; 29(1): 15-21.
30. Love M I, Huber W, Anders S. Moderated estimation of fold change and dispersion for RNA-seq data with DESeq2. Genome Biol 2014; 15(12): 550.
31. Wilk A J, Rustagi A, Zhao N Q, et al. A single-cell atlas of the peripheral immune response in patients with severe COVID-19. Nat Med 2020: 10.1038/s41591-020-0944-y.
32. Butler A, Hoffman P, Smibert P, Papalexi E, Satij a R. Integrating single-cell transcriptomic data across different conditions, technologies, and species. Nat Biotechnol 2018; 36(5): 411-20.
33. van Dijk D, Sharma R, Nainys J, et al. Recovering Gene Interactions from Single-Cell Data Using Data Diffusion. Cell 2018; 174(3): 716-29 e27.
34. Force A D T, Ranieri V M, Rubenfeld G D, et al. Acute respiratory distress syndrome: the Berlin Definition. JAMA 2012; 307(23): 2526-33.
35. Alhazzani W, Moller M H, Arabi Y M, et al. Surviving Sepsis Campaign: Guidelines on the Management of Critically Ill Adults with Coronavirus Disease 2019 (COVID-19). Crit Care Med 2020; 48(6): e440-e69.
36. Harrell F E, Jr., Lee K L, Mark D B. Multivariable prognostic models: issues in developing models, evaluating assumptions and adequacy, and measuring and reducing errors. Stat Med 1996; 15(4): 361-87.
37. Chua R, Lukassen S, Trump S, et al. Cross-talk between the airway epithelium and activated immune cells defines severity in COVID-19. MedRxiv 2020: 10.1101/2020.04.29.20084327.
38. Cecconi M, Piovani D, Brunetta E, et al. Early Predictors of Clinical Deterioration in a Cohort of 239 Patients Hospitalized for Covid-19 Infection in Lombardy, Italy. J Clin Med 2020; 9(5).
39. Immovilli P M N, Antonucci E, Radaelli G, Barbera M, Guidetti D. COVID-19 Mortality and ICU Admission: The Italian Experience. Crit Care 2020; 24(1): 228.
40. Hou H, Zhang B, Huang H, et al. Using IL-2R/lymphocytes for predicting the clinical progression of patients with COVID-19. Clin Exp Immunol 2020: 10.1111/cei.13450.
41. Chen R, Sang L, Jiang M, et al. Longitudinal hematologic and immunologic variations associated with the progression of COVID-19 patients in China. J Allergy Clin Immunol 2020: 10.1016/j.jaci.2020.05.003.
42. Yamasaki K, Kurimura M, Kasai T, Sagara M, Kodama T, Inoue K. Determination of physiological plasma pentraxin 3 (PTX3) levels in healthy populations. Clin Chem Lab Med 2009; 47(4): 471-7.
43. Ristagno G, Fumagalli F, Bottazzi B, et al. Pentraxin 3 in Cardiovascular Disease. Front Immunol 2019; 10: 823.
44. Bastrup-Birk S, Munthe-Fog L, Skjoedt M O, et al. Pentraxin-3 level at admission is a strong predictor of short-term mortality in a community-based hospital setting. J Intern Med 2015; 277(5): 562-72.
45. Hansen C B, Bayarri-Olmos R, Kristensen M K, Pilely K, Hellemann D, Garred P. Complement related pattern recognition molecules as markers of short-term mortality in intensive care patients. J Infect 2020; 80(4): 378-87.
46. Song J, Moon S, Park D W, et al. Biomarker combination and SOFA score for the prediction of mortality in sepsis and septic shock: A prospective observational study according to the Sepsis-3 definitions. Medicine (Baltimore) 2020; 99(22): e20495.
47. Katsube Y A M, Watanabe M, Abe M, Kamisago M, Fukazawa R, Ogawa S. PTX3, a new biomarker for vasculitis, predicts intravenous immunoglobulin unresponsiveness in patients with Kawasaki disease. J Am Coll Cardiol 2011; 57(14): E2038.
48. Viner R M, Whittaker E. Kawasaki-like disease: emerging complication during the COVID-19 pandemic. Lancet 2020; 395(10239): 1741-3.
49. Whittaker E, Bamford A, Kenny J, et al. Clinical Characteristics of 58 Children With a Pediatric Inflammatory Multisystem Syndrome Temporally Associated With SARS-CoV-2. JAMA 2020: 10.1001/jama.2020.10369.
50. Escher F, Pietsch H, Aleshcheva G, et al. Detection of viral SARS-CoV-2 genomes and histopathological changes in endomyocardial biopsies. ESC Heart Fail 2020: 10.1002/ehf2.12805.
51. Lodigiani C, Iapichino G, Carenzo L, et al. Venous and arterial thromboembolic complications in COVID-19 patients admitted to an academic hospital in Milan, Italy. Thromb Res 2020; 191: 9-14.

1. An in-vitro or ex vivo method for the prognosis of a Coronavirus disease and/or for the monitoring of the efficacy of a therapeutic treatment of a Coronavirus disease comprising the steps of:
a) detecting and/or measuring the amount of the protein PTX3 or of fragments thereof or of the polynucleotide coding for said protein or of fragments thereof in an isolated biological sample obtained from the subject,
preferably the method further comprises the step:
b) comparing the same with a proper control.

2. The method according to claim 1, wherein the Coronavirus is a beta Coronavirus, preferably SARS-CoV-2, and/or the Coronavirus disease is selected from the group consisting of: Severe Acute Respiratory Syndrome (SARS), Middle East Respiratory Syndrome (MERS), COVID-19, coronavirus-associated acute respiratory distress syndrome (ARDS)

3. The method according to claim 1, wherein the biological sample is selected from the group consisting of: plasma, serum, blood, CSF, saliva, or Bronchoalveolar lavage fluid (BALF) and pulmonary tissue.

4. The method according to claim 1, wherein the amount of PTX3 is detected or measured by means of specific antibody or coulometric or electrochemical detector.

5. The method according to claim 1, wherein the subject is a patient who has been diagnosed with a Coronavirus disease.

6. The method according to claim 1, wherein when PTX3 is higher than the proper control, the subject is at risk of short-term mortality or of being affected by a more severe disease and/or of a poor prognosis.

7. (canceled)

8. (canceled)

9. (canceled)

10. (canceled)

11. Kit for the prognosis of a Coronavirus disease and/or for the monitoring of the efficacy of a therapeutic treatment of a Coronavirus disease comprising:
means to detect and/or measure the amount of at least the biomarker PTX3 and optionally
control means.

12. (canceled)
